(19) United States
(12) Patent Application Publication
(10) Pub. No.: US 2020/0108039 A1
(43) Pub. Date: Apr. 9, 2020

(54) Compositions of medium chaintryglycerides (MCT) and plant-based nutrients for brain health

(71) Applicants: Robert Firger, Bloomfiled, CT (US); Gerald Haase, Denver, CO (US)

(72) Inventors: Robert Firger, Bloomfiled, CT (US); Gerald Haase, Denver, CO (US)

(21) Appl. No.: 16/350,663

(22) Filed: Dec. 19, 2018

Related U.S. Application Data
(60) Provisional application No. 62/741,646, filed on Oct. 5, 2018.

(51) Int. Cl. A61K 31/215; A61K 31/12; A61K 31/663; A61K 36/889; A61K 36/534; A61K 36/752; A61P 25/28; A61K 33/34; A61K 33/30; A61K 31/198

(52) U.S. Cl. CPC A61K 31/215; A61K 31/12; A61K 31/663; A61K 36/889; A61K 36/534; A61K 45/06; A61P 25/28; A61K 33/34; A61K 33/30; A61K 31/198; A61K 36/752

(57) ABSTRACT

A formulation that relates to enhancement of metabolic energy pathways to improve attention, cognitive, memory, analytical and executive functions of the human brain. The formulation comprising at least one medium chain triglyceride, at least one polyphenolic-rich phytonutrient, at least one brain carotenoid antioxidant, at least one dietary vitamin and mineral, and at least one nutrient, and mixtures and combinations thereof.

### RELATED APPLICATION

This application is a related to U.S. Provisional Application Ser. No. 62/741,646 filed on Oct. 5, 2018 entitled “Compositions of Medium Chain Triglycerides (MCT) and Plant-Based Nutrients for Brain Health”.

### BACKGROUND OF INVENTION

### Field of Invention

The invention relates to enhancement of metabolic energy pathways to improve attention, cognitive, memory, analytical and executive functions of the human brain. The invention utilizes a high concentration ketogenic agent formulation including dietary polyphenols and brain carotenoid antioxidants to supplement the usual biochemical pathways which generate energy in the brain and fuel and improve the execution of cognitive tasks.

### SUMMARY OF THE INVENTION

The present invention provides for a complex nutrient formulation and combination, comprising natural ketogenic agents including ingredients with high content of a caprylic/capric acid ratio, the components most effective in ketogenesis; with phenolic-rich spearmint extracts containing rosmarinic acid, marigold-derived xanthophyll carotenoid antioxidants including lutein, zeaxanthin and meso-zeaxanthin. An adjunctive nutrient formulation is provided comprising flavonoids, stilbenes, dietary and endogenous antioxidants, nicotinamide riboside, coconut oil and ketone esters. A unique proprietary method of manufacture and flavoring comprises ingredients including d-alpha tocopheryl acetate, acacia gum, E-polylysine, sodium citrate, ascorbic acid, steviol glycosides (rebaudioside A), citric acid, a flavor smoothing agent and naturally derived plant and fruit flavors and colors.

In one embodiment, a formulation comprising at least one medium chain triglyceride, at least one polyphenolic-rich phytonutrient, at least one brain carotenoid antioxidant, at least one dietary vitamin and mineral, and at least one nutrient, and mixtures and combinations thereof.

In a further embodiment wherein at least one medium chain triglyceride is selected from a group comprising MCT oil (caprylic/capric acid), ketone esters, and coconut oil extracts (caprylic, capric, lauric acid), and mixtures and combinations thereof.

In yet another embodiment, wherein at least one polyphenolic-rich phytonutrient is selected from a group comprising spearmint, citrus flavonoids (anthocyanins, bergamot, chokeberry), Grape seed (extracts, stilbenes, resveratrol), Tea (black, green, Moringa), and mixtures and combinations thereof.

In still another embodiment, wherein at least one brain carotenoid antioxidant is selected from a group comprising Lutein, Zeaxanthin, Meso-zeaxanthin, Natural mixed carotenes (alpha, beta, gamma, natural sources), and mixtures and combinations thereof.

In a further embodiment, wherein at least one dietary vitamin and mineral is selected from a group comprising Vitamin B3 (niacinamide ascorbate, yeast sources), Nicotinamide riboside (nicotinamide adenine dinucleotide NAD+, NADH), Vitamin C (calcium ascorbate, citrus, rose or berry products), Vitamin E (vegetable, wheat germ products or natural tocopherol, d-alpha tocopheryl acetate, d-alpha tocopheryl succinate), Zinc (glycinate, gluconate, oxide, sulphate and natural forms), Copper (cupric oxide and natural forms), and mixtures and combinations thereof.

In another embodiment, wherein at least one nutrient is selected from a group comprising, Fatty acids (omega-3, omega-6, flax seed extract or oil), E-polylysine, Acacia gum, Sodium citrate, Smoothenol-3108, Steviol glycosides (rebaudioside A, leaf, extract, powder), Citric acid and mixtures and combinations thereof.

In yet another embodiment, wherein medium chain triglycerides comprise 15 gms of MCT oil (caprylic/capric acid), 25 gms of ketone esters, and 25 gms of coconut oil extracts (caprylic, capric, lauric acid).

In still another embodiment, wherein polyphenolic-rich phytonutrients comprise 900 mg of spearmint, 1000 mg of citrus flavonoids (anthocyanins, bergamot, chokeberry), 250 mgs of Grape seed (extracts, stilbenes, resveratrol), 500 mg of Tea (black, green, Moringa).

In a further embodiment, wherein brain carotenoid antioxidants comprise 10 mg of Lutein, 2 mg of Zeaxanthin, 2 mg of Meso-zeaxanthin, and 15 mg of Natural mixed carotenes (alpha, beta, gamma, natural sources).

In yet another embodiment, wherein dietary vitamins and minerals comprises 20 mg of Vitamin B3 (niacinamide ascorbate, yeast sources), 500 mg of Nicotinamide riboside (Niagen® nicotinamide adenine dinucleotide NAD+, NADH), 500 mg of Vitamin C (calcium ascorbate, citrus, rose and berry products), 105 IU of Vitamin E (vegetable, wheat germ products or natural tocopherol, d-alpha tocopheryl acetate, d-alpha tocopheryl succinate), 15 mg of Zinc (glycinate, gluconate, oxide, sulphate and natural forms), and 2 mg of Copper (cupric oxide and natural forms).

In a further embodiment, wherein nutrients comprise 1000 mg of Fatty acids (omega-3, omega-6, flax seed extract or oil), 35.7 mg of E-polylysine, 7.5 gms of Acacia gum, 200 mg of Sodium citrate, 225 mg of Smoothenol-3108, 150 mg of Steviol glycosides (rebaudioside A, leaf, extract, powder), and 35 mg of Citric acid.

In still yet another embodiment, further comprising natural preservatives, flavoring, and mixtures thereof.

In another embodiment, the formulation is used for neurological health.

In still another embodiment, the formulation comprising:

MCT oil (caprylic/capric acid) in a range from about 1 to about 50 grams;
Ketone esters in a range from about 5 to 50 grams;
Coconut oil extracts (caprylic, capric, lauric acid) in a range from about 1 to about 100 grams;
Spearmint in a range from about 600 to about 1800 mg;
Citrus flavonoids (anthocyanins, bergamot, chokeberry) in a range from about 500 to about 2500 mg;
Grape seed (extracts, stilbenes, resveratrol) in arrange from about 50 to about 500 mg;
Tea (black, green, Moringa) in a range from about 50 to about 1000 mg;
Lutein in a range from about 6 to about 20 mg;
Zeaxanthin in a range from about 0.1 to about 5 mg;
Meso-zeaxanthin in a range from about 0.1 to about 5 mg;
Natural mixed carotenes (alpha, beta, gamma, natural sources) in a range from about 1 to about 60 mg;
Vitamin B3 (niacinamide ascorbate, yeast sources) in a range from about 1 to about 50 mg;
Nicotinamide riboside (Niagen® nicotinamide adenine dinucleotide, NAD+, NADH) in a range from about 100 to about 2000 mg;
Vitamin C (calcium ascorbate, citrus, rose or berry products) in a range from about 100 to about 2000 mg;
Vitamin E (vegetable, wheat germ products or natural tocopherol)
(d-alpha tocopheryl acetate) in a range from about 1 to about 200 IU;
(d-alpha tocopheryl succinate) in a range from about 25 to about 400 IU;
   Zinc (glycinate, gluconate, oxide, sulphate or natural forms) in a range from about 1 to about 80 mg;
   Copper (cupric oxide or natural forms) in a range from about 0.1 to about 4 mg;
   Fatty acids (omega-3, omega-6, flax seed extract or oil) in a range from about 100 to about 5000 mg;
   E-polylysine in a range from about 10 to about 100 mg;
   Acacia gum in a range from about 1 to about 15 grams;
   Sodium citrate in range from about 50 to about 400 mg;
   Smoothenol-3108 in a range from about 50 to about 500 mg;
   Steviol glycosides in a range from about 10 to about 250 mg;
   Citric acid in a range from about 5 to about 100 mg;
   and mixtures and combinations thereof.

In still another embodiment, a method of manufacturing a formulation, the method comprising admixing at least one medium chain triglyceride, at least one polyphenolic-rich phytonutrient, at least one brain carotenoid antioxidant, at least one dietary vitamin and mineral, and at least one nutrient, or any mixtures of combination thereof in varying amounts.

In yet a further embodiment, a method wherein said formulation is utilized for neurological health.

In another embodiment, a method wherein at least one medium chain triglyceride is selected from a group comprising MCT oil (caprylic/capric acid), ketone esters, and coconut oil extracts (caprylic, capric, lauric acid), and mixtures and combinations thereof.

In still a further embodiment, a method of wherein at least one polyphenolic-rich phytonutrient is selected from a group comprising spearmint, citrus flavonoids (anthocyanins, bergamot, chokeberry), Grape seed (extracts, stilbenes, resveratrol), Tea (black, green, Moringa), and mixtures and combinations thereof.

In another embodiment, a method of wherein at least one brain carotenoid antioxidant is selected from a group comprising Lutein, Zeaxanthin, Meso-zeaxanthin, Natural mixed carotenes (alpha, beta, gamma, natural sources), and mixtures and combinations thereof.

In still another embodiment, a method wherein at least one dietary vitamin and mineral is selected from a group comprising Vitamin B3 (niacinamide ascorbate, yeast sources), Nicotinamide riboside (nicotinamide adenine dinucleotide NAD+, NADH), Vitamin C (calcium ascorbate, citrus, rose or berry products), Vitamin E (vegetable, wheat germ products or natural tocopherol, d-alpha tocopheryl acetate, d-alpha tocopheryl succinate), Zinc (glycinate, gluconate, oxide, sulphate and natural forms), Copper (cupric oxide and natural forms), and mixtures and combinations thereof.

In a further embodiment, a method wherein at least one nutrient is selected from a group comprising, Fatty acids (omega-3, omega-6, flax seed extract or oil), E-polylysine, Acacia gum, Sodium citrate, Smoothenol-3108, Steviol glycosides (rebaudioside A, leaf, extract, powder), Citric acid and mixtures and combinations thereof.

All categories of ingredients and ingredient sources herein are illustrative only and may include named ingredients, whether or not proprietary to the current invention, as well as alternative sources and iterations of such ingredients and ingredient categories, which may be substituted, added or may be novel to the present invention.

### BRIEF DESCRIPTION OF THE DRAWINGS

The accompanying drawings are included to provide a further understanding of the present invention. These drawings are incorporated in and constitute a part of this specification, illustrate one or more embodiments of the present invention and together with the description, serve to explain the principles of the present invention.

FIG. 1 is a diagram of how ketone metabolism functions; and

FIG. 2 is a diagram of energy production in mitochondrion.

Among those benefits and improvements that have been disclosed, other objects and advantages of this invention will become apparent from the following description taken in conjunction with the accompanying drawings. The drawings constitute a part of this specification and include exemplary embodiments of the present invention and illustrate various objects and features thereof.

### DETAILED DESCRIPTION OF THE INVENTION

As required, detailed embodiments of the present invention are disclosed herein; however, it is to be understood that the disclosed embodiments are merely exemplary of the invention that may be embodied in various forms. The figures are not necessarily to scale, some features may be exaggerated to show details of particular components. Therefore, specific structural and functional details disclosed herein are not to be interpreted as limiting, but merely as a basis for the claims and as a representative basis for teaching one skilled in the art to variously employ the present invention.

### Ketogenic Agents

Numerous orally administered so-called “health” and “brain” supplements are marketed to allegedly provide additional or more effective “energy” to the brain and allow it to function in a more efficient or superior fashion. Virtually all of these supplements contain one or more of numerous ingredients, including by examples only from an exhaustive list, caffeine, sugar, acetyl-1-carnitine, vinpocetine, omega-3 fatty acids, phostatidylserine, and a variety of herbal ingredients such as green tea extracts, bacopa manniera, rhodiola rosea, Siberian ginseng, huperzia serrata, gingko biloba and various minerals and vitamins. While some or many of these ingredients may in other contexts have been shown anecdotally to have collateral uses, none has ever been shown to produce actual energy in the brain. In addition, some or many of these ingredients are destroyed during intestinal digestion. Particularly in the instance of those claiming to directly energize the brain, they do not cross the blood-brain barrier (BBB) and, thus, have no actual or potential benefit to produce actual energy in the mitochondrion.

The human brain functions energetically on only two sources of fuel, either primarily glucose or alternatively ketone bodies. The latter is an evolutionarily selected alternative source of energy in times of starvation, disease, stress or short supplies of food. Cognitive performance has been shown to diminish with advancing age. Various brain imaging studies have demonstrated that even in healthy individuals the brain (or important parts thereof) shows gradual diminished capacity to process glucose and in varying degrees results in a hypo-metabolic state. This is apparently because of a gradually developing inhibition of glucose uptake by neurons, and/or because of progressive inability of neurons to process glucose efficiently. As a result, energy deprived neurons may sicken, lose function and ultimately die. Some significant research studies have even characterized this condition as a “Type 3 Diabetes”. Ketone bodies (hydroxybutyrate and acetoacetate), produced by the liver in increasing amounts when dietary carbohydrate is in short supply, serve as an alternative energy source for central nervous system (CNS) neurons. Under “normal” conditions (when the diet provides an “adequate” supply of usable carbohydrate) glucose is the fuel used by the brain. However, when dietary carbohydrate (and therefore glucose) is in short supply (e.g. during starvation), ketones provide backup fuel for the brain. When ketone bodies are available to the brain, the brain readily uses them for energy, in fact in a mandatory fashion with ketones always being metabolized when available. Comprehensive reviews of the therapeutic potential of ketone bodies have been published.

In a variety of neurological and metabolic conditions, including those of the elderly with cognitive impairments, the aging brain is unable to access or process glucose efficiently. Ketones can help fill the resulting energy gap and thereby help restore the ability of fuel-depleted mitochondria in key brain cells to function normally. In this way, the cognitive/memory functions that depend on mitochondria-generated power (akin to electric power for the cell) can be restored to a more normal state. If such restoration is not possible owing to neuronal loss, the rate at which neurodegeneration occurs can still be arrested or slowed.

Although glucose metabolism may be a primary source of brain energy, ketone metabolism provides an alternative pathway. MCT generated ketone bodies are used by neurons as an alternative energy source to alleviate the deficit in glucose metabolism. MCT supplementation has been found to provide cognitive improvement correlated positively with the blood levels of beta hydroxybutyrate. Thus ketones, provided indirectly by consumption of medium chain triglycerides raise blood ketone concentrations sufficiently to thereby provide the energy starved brain with an alternative, more readily accessible fuel source.

It has been known for more than half a century that ingestion of medium chain triglycerides (MCT) results in a rise in blood ketones that is a ketogenic effect. As early as 1966, Vanitallie and others described the ketogenic effects of MCT in peer-reviewed journals. Many other medical uses of MCT have been reported in scientific literature over the ensuing years.

### Ketones and Cognitive Performance.

Dietary supplementation with medium chain triglycerides (MCTs) may have long-lasting cognition enhancing effects. Data supports the clinical observation that age associated reduction in cerebral glucose metabolism is a common feature in aging and diseases of older adults. The process involved is progressive and starts in early middle age or sooner. Particularly in instances of brain and metabolic pathologies, the resultant metabolic decline contributes to the cognitive declines observed as associated with aging or disease, at even earlier ages.

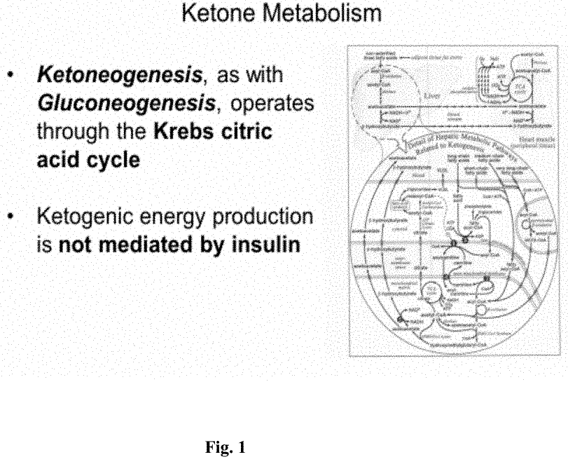

Over recent years, a ketogenic diet has been tested as a means for mitigating this further damage. The ketogenic diet has been in clinical use for more than 80 years, primarily for the symptomatic treatment of epilepsy. It is a high fat content diet in which carbohydrates are nearly eliminated so that the body has minimal dietary sources of glucose. During high rates of fatty acid oxidation, large amounts of acetyl-CoA are generated. These exceed the capacity of the TCA cycle and lead to the synthesis of three ketone bodies within liver mitochondria. Plasma levels of ketone bodies rise, with acetoacetate and β-hydroxybutyrate increasing three to four-fold or more from basal levels of 100 and 200 μM, respectively. Under conditions of low glucose, these ketone bodies are a perfectly adequate energy source for brain tissue. (see FIG. 1) Early efforts to utilize the ketogenic diet to produce a protective effect have been promising, but there remains a need to get even higher levels of ketone bodies to the affected area of the brain. Oral ketone esters have been tried for this purpose but have a very short half-life and several serious potential side-effects as well as significant palatability and digestibility issues. There is a need, therefore, for new methods for elevating cerebral ketone body concentration on a sustained basis.

### Ketogenic Agent Source Options

The invention provides effective means of truly elevating the energetic metabolism of brain tissue through introduction of supplementary ketogenic MCT's (KMCT) beyond the naturally occurring levels in human dietary processes. The present inventors have surprisingly discovered that providing ketogenic medium chain triglycerides achieves this elevation of ketone levels, when provided in certain proprietary formulations and manufactured with proprietary methods. KMCT are metabolized in the liver to provide a rich source of ketone bodies, which can be metabolized as a carbon and energy source for the body, especially the brain. Unlike ketogenic dieting, providing exogenous KMCT does not result in reduced glucose concentrations or other physiological effects associated with ketogenic dieting, and does not suffer from similar patient compliance issues. Ingestion of KMCT has no reported serious side effects and only minor and usually transitory reported effects of gastro-intestinal distress or sensitivity in some users, which has been shown to diminish with continued use.

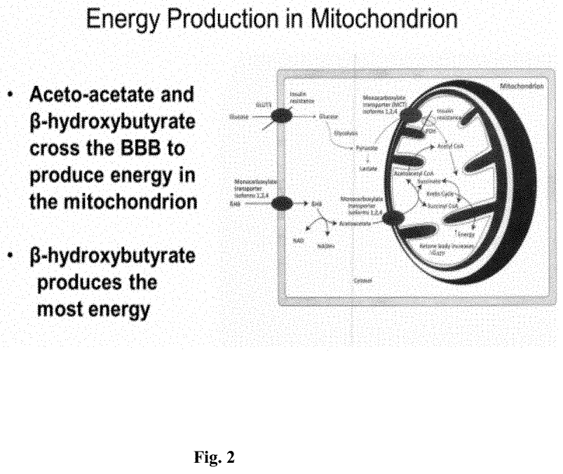

Among others, one optional convenient source for such KMCT is Fuel for Thought® (Ultimate Brain Nutrients, LLC) an orally bioavailable nutritional supplement comprising primarily caprylic triglycerides, together with additional ingredients as described herein. (see FIG. 2)

### Ketone Bodies, Esters, Salts

Ketone bodies generated in the liver from fatty acids are a potential energy source for the human body, with particular affinity and efficacy in brain and heart tissue. Increasing the blood level of ketone bodies directly has been shown to improve both cognitive and physical performance. The ketone bodies, hydroxybutyrate and acetoacetate, can be consumed to elevate ketone levels, however, the direct consumption of these compounds is difficult and potentially dangerous. For example, direct administration of these compounds may produce a dangerous medical condition known as “acidosis”.

Ketone esters can, however, be consumed together with and in combination with other compounds and ingredients as proposed in the present patent. In addition, the salts of beta-hydroxybutyrate taken with medium chain fatty acids or (medium chain triglycerides) will induce ketosis, resulting in improvements in clinical metabolic indices for insulin resistance, diabetes; as well as physical performance (providing both additional energy and lowering the limiting effects of lactic acid production) and weight management. These factors additionally contribute to the favorable consequences to cognitive measures shown by the underlying ketogenic effect of MCT's.

Through sophisticated flavor chemistry the present inventors have additionally addressed the problem of intrinsic taste and palatability difficulties of oral administration of ketones.

The present invention will utilize ketone esters and salts in varying formulas, as individual additives and in combinations and racemic mixtures. Sources may vary, as these ingredient products are newly approved and coming to market. In addition, the present invention may include new and novel variations on existing ketone esters and ketone salts and in novel combinations.

### Coconut Oil Extracts (Caprylic, Capric, Lauric Acid)

The direct ingestion of coconut oil has ketogenic activity, due to the presence of medium chain triglycerides. The MCT's in coconut oil are generally easily digested; are converted in the liver to ketones beneficial to brain activity; and may additionally have thermogenic effects (fat burning) with the MCT, lauric acid having been shown to lower the hormone Ghrelin (reducing hunger) through elevated ketone production.

Coconut oils contain approximately 60% MCTs (composed of 48% lauric acid; 6.8%; caprylic and 6.6% capric acid). Lauric acid converts to monolaurin in the human body which reports show acts as an antiviral; and capric acid and caprylic acid, having been shown to be antimicrobial lipids.

Significantly, however to the present invention, the consumption of coconut oil and its constituent MCT's improves the body's use of insulin; has been demonstrated to improve overall cholesterol by increasing HDL; and may positively affect thyroid function and assist in reduction of reactive oxygen species (ROS) implicated in many disease etiologies.

There are a large number and variety of coconut oil sources in the market. However, the quality of coconut oil is essential to its function. Many products use lower grade or undisclosed quality coconut oil ingredients (extracted with solvents and “RBD”—refined, bleached and deodorized, which is the commercial industry standard for food and nutritional products.) The present inventors intend to incorporate both whole coconut oil as well as responsibly produced extracts of coconut oil; and will utilize only the highest value cold, expeller-pressed coconut oil, containing no solvents which can otherwise reduce, impair or negate the positive effects of the coconut oil ingredients.

### Spearmint

This plant-based, high polyphenol-containing nutrient has demonstrated a variety of cognitive benefits in human clinical trials. The mechanisms of action include reduction in oxidative stress, support of neurotransmission, neurogenic induction and neuroprotection. The beneficial effects include aspects of cognition, sleep support and physical performance. Studies have shown improvement in working memory, short term memory, concentration, focus, sustained attention, falling asleep faster and support of physical multitasking, agility and reaction time.

In vitro testing has demonstrated free radical scavenging, decreased lipid peroxidation in the brain and attenuation of reactive oxygen species. Animal studies showed reduction of oxidative damage in the cerebral cortex and hippocampus. It is known that the neurotransmitter, acetylcholine, strengthens neural pathways in the brain. Importantly, the components in this phytonutrient consistently decreased cholinesterase in in vitro and in vivo experiments. Neuroprotection has also been demonstrated by downregulating apoptosis in cell line and animal studies as well as by decreasing neuronal loss and protecting mitochondrial membrane potential.

Laboratory analyses have confirmed high plasma levels of the key active phenolic molecules and their metabolites. In addition, human clinical trials have proven important clinical benefits in cognitive domains. A randomized trial of 90 subjects aged 50-70 years of age found improvement in working memory and sleep patterns. Another randomized study looked at different population and included 143 subjects aged 18-50 years of age. The trial demonstrated benefit in reactive agility on a task requiring hand and foot movements along with enhanced sustained attention.

### Spearmint Source Options.

It is critical that the plant-based extract that is selected for this invention contains an effective concentration of the key polyphenolic compounds. Among others, one option is a dried aqueous acidic extract that is prepared from two proprietary non-genetically modified lines of Mentha spicata L. This complex produces higher concentrations of phenolic molecules than what is commercially available. This product option is Neumentix™ (Kemin Foods L.C.) that contains a combination of polyphenols including rosmarinic, salvianolic A, salvianolic B, lithospermic and caftaric acids. This method of manufacture utilizes rhizome transplanting, a technique that maintains plant clonality to enhance production. Of importance, this proprietary form of spearmint has achieved GRAS (generally recognized as safe) designation in the United States.

### Carotenoids

These substances are naturally occurring organic pigments that accumulate in the brain. In fact, this class of compounds comprise prevalent and strongly active antioxidants in the cerebral cortex and brain stem structures, including the hippocampus and the macula of the eye. In parallel with this, emerging research is documenting the role of carotenoids in promoting broad cognitive health and vision protection. Two key carotenoids are orally consumable in the diet or through supplementation. These xanthophyllic antioxidants are lutein and zeaxanthin. A third substance, meso-zeaxanthin is derived from lutein.

The carotenoids in this invention are known to effectively cross the blood brain barrier and are preferentially accumulated in the brain. The high metabolic rate that occurs in the central nervous system makes the brain particularly susceptible to oxidative stress. Lutein and zeaxanthin function as powerful antioxidants and, thus, can protect the brain from free radical damage as well as support normal function and structure.

These neuroprotective benefits have been proven in human studies. A randomized clinical trial of one year of supplementation in 51 young adults found improved cognition in the areas of complex attention, reasoning and spatial memory. Another one-year human study in 44 older individuals demonstrated increased cortical activity detected on functional MM scanning. Further investigations showed protection of the retina of the eye against harmful blue light, oxidative damage and macular degeneration. Finally, a year of supplementation in 42 older subjects led to improved cognition in regard to complex attention and cognitive flexibility.

### Carotenoid Source Options.

As with the other components of this invention, the most credible and effective brain carotenoid source is critical to the uniqueness of the ultimate formulation. Such a source would have high concentrations of lutein and zeaxanthin that can prove accumulation in the eye as detected by “macular pigment optical density” testing since this measurement is strongly correlated with tissue levels of the active nutrients in the brain. Among others, one option for an ideal product to provide the necessary antioxidant effect has been found in FloraGLO Lutein® (Kemin Foods, L.C.). This complex is derived from a proprietary hybrid of the marigold plant and contains a consistent concentration of zeaxanthin at 0.83 mg for each 10 mg of lutein. Supplementation provides a sustained more than doubling of the plasma lutein levels. The robust product stability maintains tissue content for months. The U.S. Pat. No. 9,226,940 B2 issued for this complex documents the formulation's protection against the potentially harmful exposure from sunlight, computers and smart phone screens. This carotenoid combination is the most researched lutein product in the world with a more than 25-year safety record. It was utilized and proved beneficial by the U. S Government's National Eye Institute (of the NIH) in the landmark eye health supplement trial, AREDS2.

### Nicotinamide Riboside

Nicotinamide riboside (NR) is a unique member of the vitamin B3 family, that functions as a precursor to nicotinamide adenine dinucleotide or NAD+. Cells can use NR to create nicotinamide adenine dinucleotide (NAD+), which is essential to the function of all living cells.

NAD+ is a requisite in enabling cells to produce energy in the mitochondria and in conjunction with an energy source such as ketones operates in a synergistic manner. (See diagram on page . . . ). NAD+ assists in the health of mitochondria and is essential to cellular repair. It is also an important substrate for many NAD+-consuming enzymes including the sirtuins, with potential beneficial effects on aging and circadian rhythms; and PARPs (Poly (ADP-ribose) polymerase) a family of proteins involved in essential cellular functions including genomic stability, DNA repair and apoptosis (programmed cell death).

Extensive research studies show that NAD+ decreases in advancing age over time and under metabolic stress. Supplementation of NR has been demonstrated to help support many aspects of healthy aging, including cardiovascular and brain health. It also helps generate energy in mitochondrial-dense tissues like muscle, brain, and liver.

In 2016 the FDA granted generally recognized as safe (GRAS) status for nicotinamide riboside as a food ingredient in nutritional products. The present inventors intend to incorporate Nicotinamide Riboside in the adjunctive package of ingredients of the present invention to produce as “complete” a system of energy supportive, generative and associated ingredients as possible. Among others, one option for an ideal product to provide the necessary NAD+ effect has been found in Tru Niagen™ (Chromadex Corporation).

### Product Formulation

This invention provides novel formulations for brain health that are derived from five entirely separate categories of ingredients:

a) Ketogenic agents

b) Polyphenolic-rich phytonutrients

c) Brain carotenoid antioxidants

d) Dietary vitamins and minerals

e) Other nutrients

The combinations included in the overall formulation presented may vary within each category. To provide another enhancement to the unique nature of this invention, certain individuals may benefit from ingredient consumption that differs from the primary dose listed but is generally encompassed within the dose ranges. Supplementation may entail none of the ingredients from any one or more particular categories, all of the ingredients in a category or partial combinations from multiple categories thereof. The doses listed are intended to be considered the total daily dose. For commercial application, this dosage may be consumed at one time or in multiple divided doses throughout the day.

The product platform for humans includes but is not limited to concentrated liquid beverage forms in two to two and one half ounce sizes; more dilute liquid beverage forms in eight to twenty ounce sizes; drops; powders; and multiple solid formats including pills, capsules, soft gels, tablets, bars, gummies, lozenges/troches, dissolvable disks, chewables, patches, sticks; as well as aerosols, ointments, gels, and injectable, liposomal, microencapsulation, nanotechnology or other delivery systems. The standard risk formulation is intended for consumers ages twelve years and older.

### Standard Risk Formulation

| | Dosage: | | |
|---|---|---|---|
| | Primary | | Range |
| Ketogenic Agents | | | |
| MCT oil (caprylic/capric acid, other | 15 | grams | (1-50 grams) |
| sources) | | | |
| Ketone esters | 25 | grams | (5-50 grams) |
| Coconut oil extracts (caprylic, capric, | 25 | grams | (1-100 grams) |
| lauric acid) | | | |
| Polyphenolic-Rich Phytonutrients | | | |
| Spearmint (Neumentix ™ proprietary | 900 | mg | (600-1800 mg) |
| extract, other sources) | | | |
| Citrus flavonoids (anthocyanins, | 1000 | mg | (500-2500 mg) |
| bergamot, chokeberry) | | | |
| Grape seed (extracts, stilbenes, | 250 | mg | (50-500 mg) |
| resveratrol) | | | |
| Tea (black, green, Moringa) | 500 | mg | (50-1000 mg) |
| Brain Carotenoid Antioxidants | | | |
| Lutein (FloraGLO ® proprietary | 10 | mg | (6-20 mg) |
| extract, other sources) | | | |
| Zeaxanthin (FloraGLO ® proprietary | 2 | mg | (0.1-5 mg) |
| extract, other sources) | | | |
| Meso-zeaxanthin | 2 | mg | (0.1-5 mg) |
| Natural mixed carotenes (alpha, beta, | 15 | mg | (l-60 mg) |
| gamma, natural sources) | | | |
| Dietary Vitamins and Minerals | | | |
| Vitamin B3 (niacinamide ascorbate, | 20 | mg | (l-50 mg) |
| yeast sources) | | | |
| Nicotinamide riboside (Niagen ® | 500 | mg | (100-2000 mg) |
| nicotinamide adenine dinucleotide: | | | |
| NAD+, NADH, other sources) | | | |
| Vitamin C (calcium ascorbate, citrus, | 500 | mg | (100-2000 mg) |
| rose or berry products) | | | |
| Vitamin E (vegetable, wheat germ | | | |
| products or natural tocopherol) | | | |
| (d-alpha tocopheryl acetate) | 5 | IU | (1-200 IU) |
| (d-alpha tocopheryl succinate) | 100 | IU | (25-400 IU) |
| Zinc (glycinate, gluconate, oxide, | 15 | mg | (l-80 mg) |
| sulphate or natural forms) | | | |
| Copper (cupric oxide or natural | 2 | mg | (0.1-4 mg) |
| forms) | | | |
| Other Nutrients | | | |
| Fatty acids (omega-3, omega-6, flax | 1000 | mg | (100-5000 mg) |
| seed extract or oil) | | | |
| E-polylysine | 37.5 | mg | (10-100 mg) |
| Acacia gum | 7.5 | grams | (1-15 grams) |
| Sodium citrate | 200 | mg | (50-400 mg) |
| Smoothenol-3108 | 225 | mg | (50-500 mg) |
| Steviol glycosides (rebaudioside A, | 150 | mg | (10-250 mg) |
| leaf, extract, powder) | | | |
| Citric acid | 35 | mg | (5-100 mg) |

For commercial use, it is intended that this product formulation may contain the following ingredients per category:

a) one or more of three ingredients in the “Ketogenic Agents” category

b) one or more of four ingredients in the “Polyphenolic-Rich Phytonutrients” category

c) one or more of four ingredients in the “Brain Carotenoid Antioxidants’ category

d) use of ingredients in the “Dietary Vitamins and Minerals” category is optional

e) one or more of seven ingredients in the “Other Nutrients” category

### Method and Order of Manufacture

A method of manufacturing a formulation, the method comprising admixing at least one medium chain triglyceride, at least one polyphenolic-rich phytonutrient, at least one brain carotenoid antioxidant, at least one dietary vitamin and mineral, and at least one nutrient, or any mixtures of combination thereof in varying amounts.

### Impact of the Invention

The invention comprises administering to an individual an amount of one or more precursor of ketone bodies, in the form of ketogenic agents including but not limited to MCT's, sufficient to raise the extracellular cerebral concentration of ketone bodies to at least about 1 to 10 mM. In some embodiments, the precursor is a medium chain triglyceride. In some embodiments, the precursor may itself be esterified to ketone bodies. In some embodiments, the precursor may be used in combination with a ketogenic diet.

The dosages and dose ranges in this invention must have wide applicability since less than one hundred percent of oral nutrient supplements are generally fully absorbed. Therefore, it is rational to have usage discretion and dose flexibility in each ingredient category. The primary doses and ranges are designed to be at adequately broad and sufficient levels to impart the desired beneficial effects on brain health in humans of both genders and of a wide range of ages and weights. The cited research demonstrates that the nutrient categories to which the components of the present invention belong are known to provide true brain energy, enhance a spectrum of cognitive functions, reduce oxidative damage, are neuroprotective and, thereby, enhance overall brain health.

1. A formulation consisting of at least one triglyceride, at least one polyphenolic-rich phytonutrient, at least one brain carotenoid antioxidant, at least one dietary vitamin and mineral, at least one nutrient, at least one preservative, at least one flavoring agent and mixtures and combinations thereof, said triglyceride is selected from a group consisting of MCT oil, ketone esters, and coconut oil extracts, and mixtures and combinations thereof, said polyphenolic-rich phytonutrient is selected from a group consisting of spearmint, citrus flavonoids, Grape seed, Tea, and mixtures and combinations thereof, brain carotenoid antioxidant is selected from a group consisting of Lutein, Zeaxanthin, Meso-zeaxanthin, Natural mixed carotenes, and mixtures and combinations thereof, dietary vitamin and mineral is selected from a group consisting Vitamin B3, Nicotinamide riboside, Vitamin C, Vitamin E, Zinc, Copper, and mixtures and combinations thereof, said nutrient is selected from a group consisting of Fatty acids, E-polylysine, Acacia gum, Sodium citrate, Smoothenol-3108, glycosides, Citric acid and mixtures and combinations thereof.

2. (canceled)

3. (canceled)

4. (canceled)

5. (canceled)

6. (canceled)

7. The formulation of claim 1 wherein said triglycerides comprise 15 gms of MCT oil, 25 gms of ketone esters, and 25 gms of coconut oil extracts.

8. The formulation of claim 1 wherein said polyphenolic-rich phytonutrients comprise 900 mg of spearmint, 1000 mg of citrus flavonoids, 250 mgs of Grape seed, and 500 mg of Tea.

9. The formulation of claim 1 wherein said brain carotenoid antioxidants comprise 10 mg of Lutein, 2 mg of Zeaxanthin, 2 mg of Meso-zeaxanthin, and 15 mg of Natural mixed carotenes.

10. The formulation of claim 1 wherein said dietary vitamins and minerals comprises 20 mg of Vitamin B3, 500 mg of Nicotinamide riboside, 500 mg of Vitamin C, 105 IU of Vitamin E, 15 mg of Zinc, and 2 mg of Copper.

11. The formulation of claim 1 wherein said nutrients comprise 1000 mg of Fatty acids, 35.7 mg of E-polylysine, 7.5 gms of Acacia gum, 200 mg of Sodium citrate, 225 mg of Smoothenol-3108, 150 mg of Steviol glycosides, and 35 mg of Citric acid.

12. (canceled)

13. The formulation of claim 1 wherein said formulation is used for neurological health.

14. A formulation consisting of:
MCT oil in a range from about 1 to about 50 grams;
Ketone esters in a range from about 5 to 50 grams;
Coconut oil extracts in a range from about 1 to about 100 grams;
Spearmint in a range from about 600 to about 1800 mg;
Citrus flavonoids in a range from about 500 to about 2500 mg;
Grape seed in a range from about 50 to about 500 mg;
Tea in a range from about 50 to about 1000 mg;
Lutein in a range from about 6 to about 20 mg;
Zeaxanthin in a range from about 0.1 to about 5 mg;
Meso-zeaxanthin in a range from about 0.1 to about 5 mg;
Natural mixed carotenes in a range from about 1 to about 60 mg;
Vitamin B3 in a range from about 1 to about 50 mg;
Nicotinamide riboside in a range from about 100 to about 2000 mg;
Vitamin C in a range from about 100 to about 2000 mg;
Vitamin E
(d-alpha tocopheryl acetate) in a range from about 1 to about 200 IU;
(d-alpha tocopheryl succinate) in a range from about 25 to about 400 IU;
Zinc in a range from about 1 to about 80 mg;
Copper in a range from about 0.1 to about 4 mg;
Fatty acids in a range from about 100 to about 5000 mg;
E-polylysine in a range from about 10 to about 100 mg;
Acacia gum in a range from about 1 to about 15 grams;
Sodium citrate in range from about 50 to about 400 mg;
Smoothenol-3108 in a range from about 50 to about 500 mg;
Steviol glycosides in a range from about 10 to about 250 mg;
Citric acid in a range from about 5 to about 100 mg;
Preservatives and flavoring;
and mixtures and combinations thereof.

15. A method of manufacturing a formulation, said method comprising admixing at least one medium chain triglyceride, at least one polyphenolic-rich phytonutrient, at least one brain carotenoid antioxidant, at least one dietary vitamin and mineral, at least one nutrient, preservatives and flavoring or any mixtures of combination thereof in varying amounts, said triglyceride is selected from a group consisting of MCT oil, ketone esters, and coconut oil extracts, and mixtures and combinations thereof, said polyphenolic-rich phytonutrient is selected from a group consisting of spearmint, citrus flavonoids, Grape seed, Tea, and mixtures and combinations thereof, brain carotenoid antioxidant is selected from a group consisting of Lutein, Zeaxanthin, Meso-zeaxanthin, Natural mixed carotenes, and mixtures and combinations thereof, dietary vitamin and mineral is selected from a group consisting Vitamin B3, Nicotinamide riboside, Vitamin C, Vitamin E, Zinc, Copper, and mixtures and combinations thereof, said nutrient is selected from a group consisting of Fatty acids, E-polylysine, Acacia gum, Sodium citrate, Smoothenol-3108, glycosides, Citric acid and mixtures and combinations thereof.

16. The method of claim 15 wherein said formulation is utilized for neurological health.

17. (canceled)

18. (canceled)

19. (canceled)

20. (canceled)
